(19) United States
(12) Patent Application Publication
(10) Pub. No.: US 2006/0194999 A1
(43) Pub. Date: Aug. 31, 2006

(54) Gasoline production by olefin polymerization

(76) Inventors: Stephen H. Brown, Brussels (BE); Georges M.K. Mathys, Bierbeek (BE); Jane Chi-Ya Cheng, Bridgewater, NJ (US); Jeffrey T. Elks, Easton, PA (US); Ajit B. Dandekar, New York, NY (US); Benjamin S. Umansky, Fairfax, VA (US); Michael C. Clark, Pasadena, TX (US)

Correspondence Address: EXXONMOBIL RESEARCH AND ENGINEERING COMPANY;(formerly Exxon Research and Engineering Company), P. O. Box 900, Annandale, NJ, 08801-0900, US

(21) Appl. No.: 11/362,257

(22) Filed: Feb. 27, 2006

Related U.S. Application Data
(60) Provisional application No. 60/656,954, filed on Feb. 28, 2005.
(60) Provisional application No. 60/656,955, filed on Feb. 28, 2005.
(60) Provisional application No. 60/656,945, filed on Feb. 28, 2005.
(60) Provisional application No. 60/656,946, filed on Feb. 28, 2005.
(60) Provisional application No. 60/656,947, filed on Feb. 28, 2005.

(51) Int. Cl. C07C 2/68

(52) U.S. Cl. 585/467

(57) ABSTRACT

Solid phosphoric acid (SPA) olefin oligomerization process units may be converted to operation with a more environmentally favorable solid catalyst. The SPA units in which a light olefin feed is oligomerized to form gasoline boiling range hydrocarbon product, is converted unit to operation with a molecular sieve based olefin oligomerization catalyst comprising an MWW zeolite material. Besides being more environmentally favorable in use, the MWW based zeolites offer advantages in catalyst cycle life, selectivity and product quality. After loading of the catalyst, the converted unit is operated as a fixed-bed unit by passing the C₂-C₄ olefinic feed to a fixed bed of the MWW zeolite condensation catalyst, typically at a temperature from 150 to 250° C., a pressure not greater than 7000 kPag, usually less than 4000 kPag and a space velocity up to 30 WHSV. The gasoline boiling range product is notable for a high level of branched chain octenes resulting in high octane quality.

### CROSS REFERENCE TO RELATED APPLICATIONS

This application claims priority from U.S. application Ser. No. 60/656,954, filed 28 Feb. 2005, entitled “Gasoline Production By Olefin Polymerization”.

This application is related to co-pending applications Ser. Nos.______, ______, ______, and ______, of even date, claiming priority, respectively from applications Ser. Nos. 60/656,955, 60/656,945, 60/656,946 and 60/656,947, all filed 28 Feb. 2005 and entitled respectively, “Process for Making High Octane Gasoline with Reduced Benzene Content, “Vapor Phase Aromatics Alkylation Process”, “Liquid Phase Aromatics Alkylation Process” and “Olefins Upgrading Process”.

### FIELD OF THE INVENTION

This invention relates to light olefin polymerization for the production of gasoline boiling range motor fuel.

### BACKGROUND OF THE INVENTION

Following the introduction of catalytic cracking processes in petroleum refining in the early 1930s, large amounts of olefins, particularly light olefins such as ethylene, propylene, butylene, became available in copious quantities from catalytic cracking plants in refineries. While these olefins may be used as petrochemical feedstock, many conventional petroleum refineries producing petroleum fuels and lubricants are not capable of diverting these materials to petrochemical uses. Processes for producing fuels from these cracking off gases are therefore desirable and from the early days, a number of different processes evolved. The early thermal polymerization process was rapidly displaced by the superior catalytic processes of which there was a number. The first catalytic polymerization process used a sulfuric acid catalyst to polymerize isobutene selectively to dimers which could then be hydrogenated to produce a branched chain octane for blending into aviation fuels. Other processes polymerized isobutylene with normal butylene to form a co-dimer which again results in a high octane, branched chain product. An alternative process uses phosphoric acid as the catalyst, on a solid support and this process can be operated to convert all the C₃ and C₄ olefins into high octane rating, branched chain polymers. This process may also operate with a C₄ olefin feed so as to selectively convert only isobutene or both n-butene and isobutene. This process has the advantage over the sulfuric acid process in that propylene may be polymerized as well as the butenes and at the present time, the solid phosphoric acid [SPA] polymerization process remains the most important refinery polymerization process for the production of motor gasoline.

In the SPA polymerization process, feeds are pretreated to remove hydrogen sulfide and mercaptans which would otherwise enter the product and be unacceptable, both from the view point of the effect on octane and upon the ability of the product to conform to environmental regulations. Typically, a feed is washed with caustic to remove hydrogen sulfide and mercaptans, after which it is washed with water to remove organic bases and any caustic carryover. Because oxygen promotes the deposition of tarry materials on the catalyst, both the feed and wash water are maintained at a low oxygen level. Additional pre-treatments may also be used, depending upon the presence of various contaminants in the feeds. With the most common solid phosphoric acid catalyst, namely phosphoric acid on kieselguhr, the water content of the feed needs to be controlled carefully because if the water content is too high, the catalyst softens and the reactor may plug. Conversely, if the feed is too dry, coke tends to deposit on the catalyst, reducing its activity and increasing the pressure drop across the reactor. As noted by Henckstebeck, the distribution of water between the catalyst and the reactants is a function of temperature and pressure which vary from unit to unit, and for this reason different water concentrations are required in the feeds to different units. Petroleum Processing Principles And Applications, R. J. Hencksterbeck McGraw-Hill, 1959.

There are two general types of units used for the SPA process, based on the reactor type, the unit may be classified as having chamber reactors or tubular reactors. The chamber reactor contains a series of catalyst beds with bed volume increasing from the inlet to the outlet of the reactor, with the most common commercial design having five beds. The catalyst load distribution is designed to control the heat of conversion.

Chamber reactors usually operate with high recycle rates. The recycle stream, depleted in olefin content following polymerization, is used to dilute the olefin at the inlet of the reactor and to quench the inlets of the following beds. Chamber reactors usually operate at pressure of approximately 3500-5500 kPag (about 500-800 psig) and temperature between 180° to 200° C. (about 350°-400° F.). The conversion, per pass of the unit, is determined by the olefin specification in the LPG product stream. Fresh feed LHSV is usually low, approximately 0.4 to 0.8 hr⁻¹. The cycle length for chamber reactors is typically between 2 to 4 months.

The tubular reactor is basically a shell-and-tube heat exchanger in which the polymerization reactions take place in a number of parallel tubes immersed in a cooling medium and filled with the SPA catalyst. Reactor temperature is controlled with the cooling medium, invariably water in commercial units, that is fed on the shell side of the reactor. The heat released from the reactions taking place inside the tubes evaporates the water on the shell side. Temperature profile in a tubular reactor is close to isothermal. Reactor temperature is primarily controlled by means of the shell side water pressure (controls temperature of evaporation) and secondly by the reactor feed temperature. Tubular reactors usually operate at pressure between 5500 and 7500 kPag (800-1100 psig) and temperature of around 200° C. (about 400° F.). Conversion per pass is usually high, around 90 to 93% and the overall conversion is around 95 to 97%. The space velocity in tubular reactors is typically high, e.g., 2 to 3.5 hr⁻¹ LHSV. Cycle length in tubular reactors is normally between 2 to 8 weeks.

For the production of motor gasoline only butene and lighter olefins are employed as feeds to polymerization processes as heavier olefins up to about C₁₀ or C₁₁ can be directly incorporated into the gasoline. With the SPA process, propylene and butylene are satisfactory feedstocks and ethylene may also be included, to produce a copolymer product in the gasoline boiling range. Limited amounts of butadiene may be permissible although this diolefin is undesirable because of its tendency to produce higher molecular weight polymers and to accelerate deposition of coke on the catalyst. The process generally operates under relatively mild conditions, typically between 150° and 200° C., usually at the lower end of this range between 150° and 180° C., when all butenes are polymerized. Higher temperatures may be used when propylene is included in the feed. In a well established commercial SPA polymerization process, the olefin feed together with paraffinic diluent, is fed to the reactor after being preheated by exchange with the reaction effluent.

The solid phosphoric acid catalyst used is non-corrosive, which permits extensive use of carbon steel throughout the unit. The highest octane product is obtained by using a butene feed, with a product octane rating of [R+M]/2 of 89 to 91 being typical. With a mixed propylene/butene feed, product octane is typically about 91 and with propylene as the primary feed component, product octane drops to typically 87.

In spite of the advantages of the SPA polymerization process, which have resulted in over 200 units being built since 1935 for the production of gasoline fuel, a number of disadvantages are encountered, mainly from the nature of the catalyst. Although the catalyst is non-corrosive, so that much of the equipment may be made of carbon steel, it does lead it to a number of drawbacks in operation. First, the catalyst life is relatively short as a result of pellet disintegration which causes an increase in the reactor pressure drop. Second, the spent catalyst encounters difficulties in handling from the environmental point of view, being acidic in nature. Third, operational and quality constraints limit flexible feedstock utilization. Obviously, a catalyst which did not have these disadvantages would offer considerable operating and economic advantages.

The Mobil Olefins-to-Gasoline [MOG] process employs a proprietary shape selective zeolite catalyst in a fluidized bed reactor to produce high octane motor gasoline by the conversion of reactive olefins such as ethylene and propylene in FCC off-gas; butenes as well as higher olefins may also be included and converted to form a high octane, branched chain gasoline product. The feed is converted over the catalyst into C₅+ components by mechanisms including oligomerization, carbon number redistribution hydrogen transfer, aromatization, alkylation and isomerization. Based on olefins converted, MOG yields 60 to 75 weight percent of high-octane gasoline blend stock with specific qualities of the product depending of the processing severity selected and the character of the feed olefins. Typically, the octane rating for the product is in the range of 88 to 91 [R+M]/2. The zeolite catalyst used in the process is environmentally safe and its attrition rate is low, and as an alternative to disposal, the spent catalyst can be reused in the FCC unit to increase octane quality.

The MOG process has, however, the economic disadvantage relative to the SPA process in that new capital investment may be required for the fluidized bed reactor and regenerator used to operate the process. If an existing SPA unit is available in the refinery, it may be difficult to justify replacement of the equipment in spite of the drawbacks of the SPA process, especially in view of current margins on fuel products. Thus, although the MOG process is technically superior, with the fluidized bed operation resolving heat problems and the catalyst presenting no environmental problems, displacement of existing SPA polymerization units has frequently been economically unattractive. What is required, therefore, is an economically attractive alternative to the SPA process for the condensation of light olefins to form motor fuels. Desirably, the process should be capable of operation in existing refinery equipment, especially as a “drop in” type replacement for the solid phosphoric acid catalyst used in the SPA process so that existing SPA polymerization units can be directly used with the new catalyst. This implies that the process should use a non-corrosive, solid catalyst in fixed bed catalyst operation. Furthermore, the catalyst should present fewer handling, operational and disposal problems than solid phosphoric acid and, for integration into existing refineries, the product volumes and distributions should be comparable to those of the SPA process.

### SUMMARY OF THE INVENTION

We have now devised a process for the conversion of light olefins such as ethylene, propylene, and butylene to gasoline boiling range motor fuels which is capable of being used as a replacement for solid phosphoric acid catalyst in process units which have previously been used for the SPA process. The catalyst used in the present process is a solid, particulate catalyst which is non-corrosive, which is stable in fixed bed operation, which exhibits the capability of cycle durations before regeneration is necessary and which can be readily handled and which can be finally disposed of simply and economically without encountering significant environmental problems. Accordingly, the catalyst used in the present process commends itself as a “drop in” replacement for the solid phosphoric acid catalyst used in the SPA catalytic condensation process for the production of motor fuels.

According to the present invention, a light olefin stream such as ethylene, propylene, optionally with butylene and possibly other light olefins, is polymerized to form a gasoline boiling range [C₅+−200° C.] [C₅+−400° F.] product in the presence of a catalyst which comprises a member of the MWW family of zeolites, a family which includes zeolites PSH 3, MCM-22, MCM-49, MCM-56, SSZ 25, ERB-1 and ITQ-1. The term “polymerized” is used here consistent with the petroleum refinery usage although, in fact, the process is one of oligomerization (which term will be used in this specification interchangeably with the conventional term) in which a low molecular weight polymer is the desired product. The process is carried out in a fixed bed of the catalyst with feed dilution, normally a hydrocarbon diluent, or added quench to control the heat release which takes place. In additional to their easy handling and amenability to regeneration, the solid catalysts used in the present process exhibit better activity and selectivity than solid phosphoric acid; compared to SPA, MCM-22 itself is three to seven times more active and significantly more stable for the production of motor gasoline by the polymerization of light olefin feeds. The catalytic performance of regenerated MCM-22 catalyst is comparable to that of the fresh MCM-22 catalyst, demonstrating that the catalyst is amenable to conventional oxidative regeneration techniques.

The conversion of an SPA process unit to operation with the present molecular sieve based catalysts therefore comprises, in principle, withdrawing the solid phosphoric acid [SPA] catalyst from the unit and loading an olefin condensation catalyst comprising an MWW zeolite material into the reactor of the process unit. Following the conversion to operation with the MWW zeolite catalyst, the unit may be used for production of the gasoline and, if desired, other liquid hydrocarbon fuels by polymerization of the refinery olefins using the appropriate conditions as described below.

### DRAWINGS

FIG. 1 shows a process schematic for the olefin polymerization unit for converting light refinery olefins to motor gasoline by the present process.

### DETAILED DESCRIPTION OF THE INVENTION

SPA Unit Conversion

The present process is for the condensation of light cracking olefins to produce motor gasoline and other motor fuels, for example, road diesel blend stock and is intended to provide a replacement for the SPA polymerization process. It provides a catalyst which can be used as a direct replacement for SPA and so enables existing SPA units to be used directly with the new catalyst, so allowing the advantages of the new catalyst and process to be utilized while retaining the economic benefit of existing refinery equipment.

The process units used for the operation of the SPA process for the catalytic condensation of light olefins to produce motor fuels are well known. These units typically comprise a feed surge drum to which the olefins and any diluent are supplied, followed by a heat exchanger in which the feed is preheated by exchange with the reactor effluent, after which it is charged to the reactor where the polymerization (condensation) takes place. Control of the heat release in the reactor is accomplished both by feed dilution and by the injection of recycled quench between catalyst beds in the reactor. The reactor effluent, cooled in exchange with the feed, is directed to a flash drum where the flash vapor is condensed and the condensate cooled. Some of the condensate is recycled for use as feed diluent and quench. Flash drum liquid flows to a stabilizer where the polymer gasoline product at the desired Reid Vapor Pressure [RVP] and light ends are separated. The light ends may be sent to a C₃-C₄ splitter depending on the refinery needs. SPA units of this kind can be directly converted to use the catalysts of the present process without significant changes since the present catalysts are a straight forward “drop in” replacement for the solid phosphoric acid [SPA] catalyst used in the conventional process technology.

Like SPA, the molecular sieve catalysts used in the present process are non-corrosive but possess significant advantages with respect to SPA, in that they are more stable, less subject to break down and are largely unaffected by the amount of water in the feed. The present catalysts are readily regenerable using conventional hydrogen stripping or oxidative regeneration, after which complete catalytic activity is substantially restored. Cycle times before regeneration or reactivation is required may be six months, one year, or even longer, representing a significant improvement over SPA. Since conventional SPA condensation units necessarily include facilities for the discharge and reloading of catalysts as a result of the short life of a catalyst, these units may readily accommodate the present molecular sieve catalysts. The SPA units do not, however, include facilities for in-situ regeneration since the SPA catalyst is used on a once-through basis before it requires disposal. The molecular sieve catalysts used in the present process, however, are fully regenerable and for this purpose, will need to be withdrawn from the reactors for ex-situ regeneration. This will typically be a simple matter to arrange using the spent catalyst discharge equipment of the SPA unit. Similarly, the SPA charging equipment lends itself directly to the charging of the zeolite catalysts into the reactors.

Unit Conversion

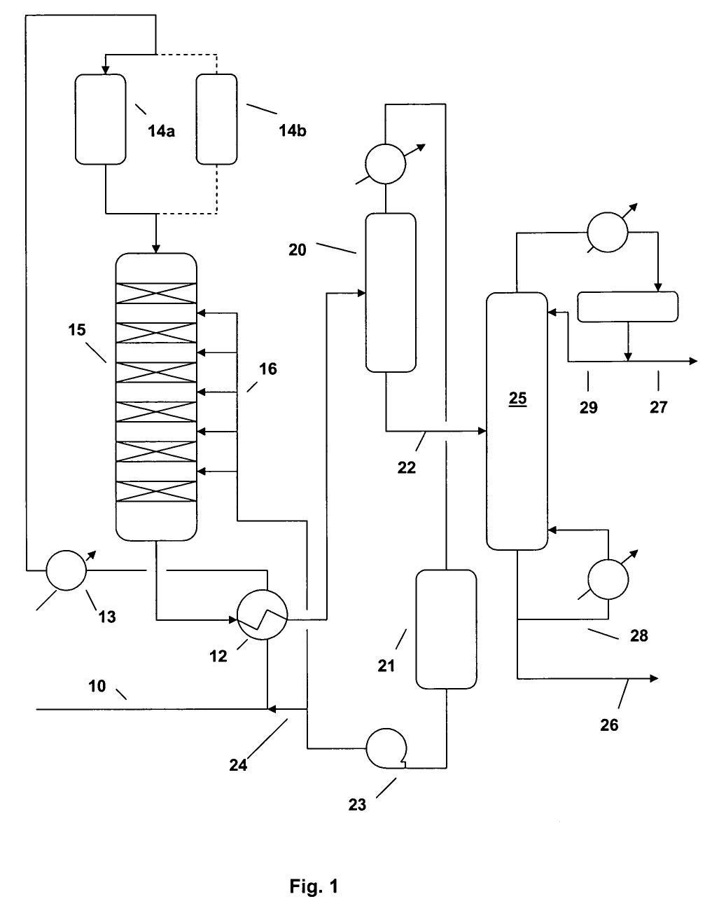

A schematic for a converted olefin condensation unit made by the conversion of an existing SPA unit is shown in simplified from in FIG. 1. A light olefin feed, typically C₂, C₃ or C₄ olefins or mixtures of these olefins from an FCC gas plant, is led into the unit through line 10 and combined with recycled hydrocarbon as diluent before passing through heat exchanger 12 in which it picks up heat from the reactor effluent before being brought to reaction temperature in heater 13. The olefin charge plus diluent passes through a guard bed reactor 14a to remove contaminants such as organic nitrogen and sulfur-containing impurities. The guard bed may be operated on the swing cycle with two beds, 14a, 14b, one bed being used on stream for contaminant removal and the other on regeneration in the conventional manner. If desired, a three-bed guard bed system may be used with the two beds used in series for contaminant removal and the third bed on regeneration. With a three guard system used to achieve low contaminant levels by the two-stage series sorption, the beds will pass sequentially through a three-step cycle of: regeneration, second bed sorption, first bed sorption.

The olefins in the charge stream are polymerized or condensed in reactor 15 to form the desired olefin polymer product during its passage over a sequence of catalyst beds in the reactor. Additional diluent is injected as quench from line 16 between the beds in order to control the exotherm. Effluent passes out of the reactor through heat exchanger 12 and then to flash drum 20 in which the diluent is separated from the olefin polymer product. The diluent which is suitably a light paraffin such as propane, butane and a portion of the polymerization product, is passed to recycle drum 21 and from there by way of recycle pump 23 and line 24 to feed line 10 for feed dilution and to recycle line 16 for injection as interbed quench in reactor 15. The olefin polymer product passes out of flash drum 20 through line 22 to the fractionator 25 to provide the final stabilized gasoline blend component in line 26 with reboil loop 28 providing column heat; light ends including unreacted olefins pass out through line 27 from reflux loop 29. As noted below, there is the potential for iso-paraffinic components to undergo reaction with the olefins in the feed to produce highly desirable branched chain reaction products of high octane value in the gasoline boiling range. The use of the recycle as feed diluent is therefore desirable not only for controlling reaction temperatures but also since it may also result in an increase in product octane.

Catalyst

The catalysts used in the present process contain, as their essential catalytic component, a molecular sieve of the MWW type. The MWW family of zeolite materials has achieved recognition as having a characteristic framework structure which presents unique and interesting catalytic properties. The MWW topology consists of two independent pore systems: a sinusoidal ten-member ring [10 MR] two dimensional channel separated from each other by a second, two dimensional pore system comprised of 12 MR super cages connected to each other through 10 MR windows. The crystal system of the MWW framework is hexagonal and the molecules diffuse along the [100] directions in the zeolite, i.e., there is no communication along the c direction between the pores. In the hexagonal plate-like crystals of the MWW type zeolites, the crystals are formed of relatively small number of units along the c direction as a result of which, much of the catalytic activity is due to active sites located on the external surface of the crystals in the form of the cup-shaped cavities. In the interior structure of certain members of the family such as MCM-22, the cup-shaped cavities combine together to form a supercage. The MCM-22 family of zeolites has attracted significant scientific attention since its initial announcement by Leonovicz et al. in Science 264, 1910-1913 [1994] and the later recognition that the family is currently known to include a number of zeolitic materials such as PSH 3, MCM-22, MCM-49, MCM-56, SSZ-25, ERB-1, ITQ-1, and others. Lobo et al. AlChE Annual Meeting 1999, Paper 292J.

The relationship between the various members of the MCM-22 family have been described in a number of publications. Four significant members of the family are MCM-22, MCM-36, MCM-49, and MCM-56. When initially synthesized from a mixture including sources of silica, alumina, sodium, and hexamethylene imine as an organic template, the initial product will be MCM-22 precursor or MCM-56, depending upon the silica:alumina ratio of the initial synthesis mixture. At silica:alumina ratios greater than 20, MCM-22 precursor comprising H-bonded vertically aligned layers is produced whereas randomly oriented, non-bonded layers of MC-56 are produced at lower silica:alumina ratios. Both these materials may be converted to a swollen material by the use of a pillaring agent and on calcination, this leads to the laminar, pillared structure of MCM-36. The as-synthesized MCM-22 precursor can be converted directly by calcination to MCM-22 which is identical to calcined MCM-49, an intermediate product obtained by the crystallization of the randomly oriented, as-synthesized MCM-56. In MCM-49, the layers are covalently bonded with an interlaminar spacing slightly greater than that found in the calcined MCM-22/MCM 49 materials. The unsynthesized MCM-56 may be calcined itself to form calcined MCM 56 which is distinct from calcined MCM-22/MCM-49 in having a randomly oriented rather than a laminar structure. In the patent literature MCM-22 is described in U.S. Pat. No. 4,954,325 as well as in U.S. Pat. Nos. 5,250,777; 5,284,643 and 5,382,742. MCM-49 is described in U.S. Pat. No. 5,236,575; MCM-36 in U.S. Pat. No. 5,229,341 and MCM-56 in U.S. Pat. No. 5,362,697.

The preferred zeolitic material for use in the catalyst of the present process is MCM-22 although zeolite MCM-49 may be found to have certain advantages relative to MCM-22. It has been found that the MCM-22 may be either used fresh, that is, not having been previously used as a catalyst or alternatively, regenerated MCM-22 may be used. Regenerated MCM-22 may be used after it has been used in any of the catalytic processes for which it is suitable, including the present process in which the catalyst has shown itself remain active after even multiple regenerations. It may also be possible to use MWW catalysts which have previously been used in other commercial processes and for which they are no longer acceptable, for example, MCM-22 catalyst which has previously been used for the production of aromatics such as ethylbenzene or cumene, normally using reactions such as alkyaltion and transalkylation. The cumene production (alkylation) process is described in U.S. Pat. No. 4,992,606 (Kushnerick et al). Ethylbenzene production processes are described in U.S. Pat. Nos. 3,751,504 (Keown); 4,547,605 (Kresge); and 4,016,218 (Haag); U.S. Pat. Nos. 4,962,256; 4,992,606; 4,954,663; 5,001,295; and 5,043,501 describe alkylation of aromatic compounds with various alkylating agents over catalysts comprising MWW zeolites such as PSH-3 or MCM-22. U.S. Pat. No. 5,334,795 describes the liquid phase synthesis of ethylbenzene with MCM-22. As noted above, MCM-22 catalysts may be regenerated after catalytic use in these processes and other aromatics production processes by conventional air oxidation techniques similar to those used with other zeolite catalysts. Conventional air oxidation techniques are also suitable when regenerating the catalysts after use in the present process.

In addition to the MWW active component, the catalysts for use in the present process will often contain a matrix material or binder in order to give adequate strength to the catalyst as well as to provide the desired porosity characteristics in the catalyst. High activity catalysts may, however, be formulated in the binder-free form by the use of suitable extrusion techniques, for example, as described in U.S. Pat. No. 4,908,120. When used, matrix materials suitably include alumina, silica, silica alumina, titania, zirconia, and other inorganic oxide materials commonly used in the formulation of molecular sieve catalysts. For use in the present process, the level of MCM-22 in a finished matrixed catalyst will be typically from 20 to 70% by weight, and in most cases from 25 to 65% by weight. In manufacture of a matrixed catalyst, the active ingredient will typically be mulled with the matrix material using an aqueous suspension of the catalyst and matrix, after which the active component and the matrix are extruded into the desired shape, for example, cylinders, hollow cylinders, trilobe, quadlobe, etc. A binder material such as clay may be added during the mulling in order to facilitate extrusion, increase the strength of the final catalytic material and to confer other desirable solid state properties. The amount of clay will not normally exceed 10% by weight of the total finished catalyst. Self-bound catalysts (alternatively referred to as unbound or binder-free catalysts), that is, catalysts which do not contain a separately added matrix or binder material, are useful and may be produced by the extrusion method described in U.S. Pat. No. 4,582,815, to which reference is made for a description of the method and of the extruded products obtained by its use. The method described there enables extrudates having high constraining strength to be produced on conventional extrusion equipment and accordingly, the method is eminently suitable for producing the high activity self-bound catalysts. The catalysts are produced by mulling the zeolite, as described in U.S. Pat. No. 4,582,815, with water to a solids level of 25 to 75 wt % in the presence of 0.25 to 10 wt % of basic material such as sodium hydroxide. Further details are to be found in U.S. Pat. No. 4,582,815. Generally, the self-bound catalysts can be characterized as particulate catalysts in the form, for instance, of extrudates or pellets, containing at least 90 wt. pct., usually at least 95 wt. pct., of the active zeolite component with no separately added binder material e.g. alumina, silica-alumina, silica, titania, zirconia etc. Extrudates may be in the conventional shapes such as cylinders, hollow cylinders, trilobe, quadlobe, flat platelets etc.

The catalyst used in the guard bed will normally be the same catalyst used in the alkylation reactor as a matter of operating convenience but this is not required: if desired another catalyst or sorbent to remove contaminants from the feed may used, typically a cheaper guard bed sorbent, e.g a used catalyst from another process or alumina. The objective of the guard bed is to remove the contaminants from the feed before the feed comes to the reaction catalyst and provided that this is achieved, there is wide variety of choice as to guard bed catalysts and conditions useful to this end. The volume of the guard bed will normally not exceed about 20% of the total catalyst bed volume of the unit.

Olefin Feed

The light olefins used as the feed for the present process are normally obtained by the catalytic cracking of petroleum feedstocks to produce gasoline as the major product. The catalytic cracking process, usually in the form of fluid catalytic cracking (FCC) is well established and, as is well known, produces large quantities of light olefins as well as olefinic gasolines and by-products such as cycle oil which are themselves subject to further refining operations. The olefins which are primarily useful in the present process are the lighter olefins from ethylene up to butene; although the heavier olefins may also be included in the processing, they can generally be incorporated directly into the gasoline product where they provide a valuable contribution to octane. The present process is highly advantageous in that it will operate readily not only with butene and propylene but also with ethylene and thus provides a valuable route for the conversion of this cracking by-product to the desired gasoline product. For this reason as well as their ready availability in large quantities in a refinery, mixed olefin streams such a FCC Off-Gas streams (typically containing ethylene, propylene and butenes) may be used. Conversion of the C₃ and C₄ olefin fractions from the cracking process provides a direct route to the branch chain C₆, C₇ and C₈ products which are so highly desirable in gasoline from the view point of boiling point and octane. Besides the FCC unit, the mixed olefin streams may be obtained from other process units including cokers, visbreakers and thermal crackers. The presence of diolefins which may be found in some of these streams is not disadvantageous since catalysis on the MWW family of zeolites takes place on surface sites rather than in the interior pore structure as with more conventional zeolites so that plugging of the pores is less problematic catalytically. Appropriate adjustment of the process conditions will enable co-condensation products to be produced when ethylene, normally less reactive than its immediate homologs, is included in the feed. The compositions of two typical FCC gas streams is given below in Tables 1 and 2, Table 1 showing a light FCC gas stream and Table 2 a stream from which the ethylene has been removed in the gas plant for use in the refinery fuel system.

TABLE 1
FCC Light Gas Stream

| Component | Wt. Pct. | Mol. Pct. |
|---|---|---|
| Ethane | 3.3 | 5.1 |
| Ethylene | 0.7 | 1.2 |
| Propane | 14.5 | 15.3 |
| Propylene | 42.5 | 46.8 |
| Iso-butane | 12.9 | 10.3 |
| n-Butane | 3.3 | 2.6 |
| Butenes | 22.1 | 18.32 |
| Pentanes | 0.7 | 0.4 |

TABLE 2
C₃-C₄ FCC Gas Stream

| Component | Wt. Pct. |
|---|---|
| 1-Propene | 18.7 |
| Propane | 18.1 |
| Isobutane | 19.7 |
| 2-Me-1-propene | 2.1 |
| 1-Butene | 8.1 |
| n-Butane | 15.1 |
| Trans-2-Butene | 8.7 |
| Cis-2-butene | 6.5 |
| Isopentane | 1.5 |
| C3 Olefins | 18.7 |
| C4 Olefins | 25.6 |
| Total Olefins | 44.3 |

While the catalysts used in the present process are robust they do have sensitivity to certain contaminants (the conventional zeolite deactivators), especially organic compounds with basic nitrogen as well as sulfur-containing organics. It is therefore preferred to remove these materials prior to entering the unit if extended catalyst life is to be expected. Scrubbing with contaminant removal washes such as caustic, MEA or other amines or aqueous wash liquids will normally reduce the sulfur level to an acceptable level of about 10-20 ppmw and the nitrogen to trace levels at which it can be readily tolerated. One attractive feature about the present process is that although activity benefits are achieved by the use of low or very low water levels in the feed, it is not otherwise unduly sensitive to water, making it less necessary to control water entering the reactor than it is in SPA units. Unlike SPA, the zeolite catalyst does not require the presence of water in order to maintain activity and therefore the feed may be dried before entering the unit, for example, to below 200 ppmw water or lower, e.g. below 50 or even 20 ppmw. In conventional SPA units, the water content typically needs to be held between 300 to 500 ppmw at conventional operating temperatures for adequate activity while, at the same time, retaining catalyst integrity. The present zeolite catalysts, however, may readily tolerate higher levels of water up to about 1,000 ppmw water although levels above about 800 ppmw may reduce activity, depending on temperature. Thus, with converted units, the olefin feed may contain from 300 or 500 to 1,000 ppmw water, although 300-800 ppmw should be regarded as a workable range for activity with existing feed treatment equipment. As noted, however, activity benefits are secured with markedly lower feed water levels and these benefits may justify feed pre-treatment to operate at lower water contents.

The use of the guard bed is particularly desirable in the operation of the present process since the refinery feeds customarily routed to polymerization units (as distinct from petrochemical unit feeds which are invariably high purity feeds for which no guard bed is required) may have a contaminant content, especially of polar catalyst poisons, such as the polar organic nitrogen and organic sulfur compounds, which is too high for extended catalyst life. The use of a cheaper catalyst in the guard bed reactors which can be readily regenerated in swing cycle operation or, alternatively disposed of on a once-through basis, is therefore desirable in ensuring extended cycle duration for the active polymerization catalyst.

Process Parameters

The present process is notable for its capability of being operated at low temperatures and under moderate pressures. In general, the temperature will be from about 120° to 250° C. (about 250° to 480° F.) and in most cases between 150° and 250° C. (about 300°-480° C.). Temperatures of 170° to 205° C. (about 340° to 400° F.) will normally be found optimum for feeds comprising butene while higher temperatures will normally be appropriate for feeds with significant amounts of propene. Pressures may be those appropriate to the type of unit from which the conversion was made, so that pressures up to about 7500 kPag (about 1100 psig) will be typical but normally lower pressures will be sufficient, for example, below about 7,000 Kpag (about 1,000 psig) and lower pressure operation may be readily utilized, e.g. up to 3500 kPag (about 500 psig). Ethylene, again, will require higher temperature operation to ensure that the products remain in the gasoline boiling range. Space velocity may be quite high, for example, up to 50 WHSV (hr⁻¹) but more usually in the range of 5 to 30 WHSV.

Gasoline Product Formation

With gasoline as the desired product, a high quality product is obtained from the polymerization step, suitable for direct blending into the refinery gasoline pool after fractionation as described above. With clean feeds, the product is correspondingly low in contaminants. The product is high in octane rating with RON values of 95 being regularly obtained and values of over 97 being typical; MON is normally over 80 and typically over 82 so that (RON+MON)/2 values of at least 89 or 90 are achievable with mixed propylene/butene feeds. Of particular note is the composition of the octenes in the product with a favorable content of the higher-octane branched chain components. The linear octenes are routinely lower than with the SPA product, typically being below 0.06 wt. pct. of all octenes except at the highest conversions and even then, the linears are no higher than those resulting from SPA catalyst. The higher octane di-branched octenes are noteworthy in consistently being above 90 wt. pct. of all octenes, again except at the highest conversions but in all cases, higher than those from SPA; usually, the di-branched octenes will be at least 92 wt. pct of all octenes and in favorable cases at least 93 wt. pct. The levels of tri-branched octenes are typically lower than those resulting from the SPA process especially at high conversions, with less than 4 wt. pct being typically except at the highest conversions when 5 or 6 wt. pct. of all octenes may be achieved, approximately half that resulting from SPA processing. In the C₅−200° C. product fraction, high levels of di-branched C8 hydrocarbons may be found, with at least 85 weight percent of the octene components being di-branched C8 hydrocarbons, e.g. 88 to 96 weight percent di-branched C8 hydrocarbons.

Depending on feed composition, reactions other than direct olefin polymerization may take place. If branch chain paraffins are present, for example, from the paraffinic diluent stream, olefin-isoparaffin alkylation reactions may take place, especially at the higher conversion levels to which the process is well suited, leading to the production of branched-chain, gasoline boiling range products of high octane rating. The reaction between butene and iso-butane and between propylene and iso-butane is of particular value since it will result in the product containing very desirable, high octane gasoline components. At low to moderate olefin conversion levels, the isoparaffin-olefin alkylation reaction is not significant but at higher conversions above about 75% (olefin conversion), e.g. at conversion levels of 80% or more (olefin conversion), particularly at 90% or higher, this reaction will increase markedly with the production of high octane gasoline components.

The following examples are given by way of illustration.

### EXAMPLE 1

### 2-Butene Oligomerization with Solid Phosphoric Acid

A commercial solid phosphoric acid (SPA) catalyst was sized to 14-24 mesh in a glove box. In a glove bag, one gram of this sized catalyst was diluted with sand to 3 cc and charged to an isothermal, down-flow, 9 mm (outside diameter) fixed-bed reactor. The catalyst was dried at 150° C. and atmospheric pressure with 100 cc/min flowing N₂ for 2 hours. The N₂ was turned off and reactor pressure was set to 5270 kPaa (750 psig) by a Grove loader. A 2-butene feed (containing 57.1% cis-butene, 37.8% trans-butene, 2.5% n-butane, 0.8% isobutene and 1-butene, 1.8% others) was introduced into the reactor at 60 cc/hr until desire reactor pressure of 5270 kPaa (750 psig) was reached. The reactor temperature was then ramped at 2° C. per minute to 170° C. During the temperature ramp, the feed flow was reduced to a desired level and kept at this level for 12 hours before data collection. Liquid products were collected at 50%, 70%, then 90% conversion (not necessary in this order) in a cold-trap and analyzed off-line.

Product carbon number distribution was determined with a HP-5890 GC equipped with a 60 meter DB-1 column (0.25 mm id and 1000 nm film thickness). Product branching was determined with an H₂−GC using a HP-5890 GC equipped with (a) a 100 meter DB-1 column (0.25 mm id and 500 nm film thickness); (b) hydrogen as the carrier gas; and (c) 0.1 g of 0.5% Pt/alumina catalyst in the GC insert for in-situ hydrogenation. Both GC's use the same temperature program: 2 min at −20° C., 8° C./min to 275° C., hold at 275° C. for 35 min.

Representative data collected at 50%, 70%, and 90% nominal conversion are shown in Tables 1-3.
Average Branching=0×% linear+1×% mono-branched+2×% di-branched+3×% tri-branched
where: % linear+% mono-branched+% di-branched+% tri-branched=100%

### EXAMPLE 2

### 2-Butene Oligomerization with A Binder-Free MCM-22

The catalyst was a binder-free, 1.6 mm quadrulobe extrudate containing 100% MCM-22. A 0.10 gram sample of this catalyst, chopped to 3 mm length, was tested for 2-butene oligomerization using the same procedure described in Example 1. Representative data are shown in Tables 3-5.

### EXAMPLE 3

### 2-Butene Oligomerization with Alumina-Bound MCM-22

The catalyst was a 1.6 mm cylindrical extrudate containing 65% MCM-22 and 35% alumina binder. A 0.20 gram of this catalyst, chopped to 1.6 mm length, was tested for 2-butene oligomerization using the same procedure described in Example 1. Representative data are shown in Tables 3-5.

### EXAMPLE 4

### 2-Butene Oligomerization with a Spent-Regenerated MCM-22

The same MCM-22 catalyst, as described in Example 3, was used in a commercial petrochemical catalytic process until it became unsuitable for further service in this application; it was then commercially regenerated in full air at 455° C. (850° F.) and about 60 torr partial pressure of water. The catalyst was treated at these conditions for about 1 hour to achieve complete regeneration. A 0.15 gram of this catalyst, chopped to 1.6 mm length, was evaluated for 2-butene oligomerization using the same procedure described in Example 1. Representative data are shown in Tables 3 -5.

### EXAMPLE 5

### 2-Butene Oligomerization in with a Spent-Regenerated-Steamed MCM-22

The same Spent-Regenerated MCM-22 catalyst, as described in Example 4, was steamed at 510° C. and 1 atm for 5 hours with 100% steam. A 0.20 gram of this “spent-regenerated-steamed” catalyst, chopped to 1.6 mm length, was evaluated for 2-butene oligomerization using the same procedure described in Example 1. Representative data are shown in Tables 3-5.

### EXAMPLE 6

### 2-Butene Oligomerization with a Multiply-Regenerated MCM-22

The same Spent-Regenerated MCM-22 catalyst, as described in Example 4, was further treated in the laboratory to simulate multiple commercial regeneration (oxidative) conditions. The sample was treated in flowing air at 700 v/v/minute, 455 C. (850° F.) and 60 torr partial pressure of water for 1 hour. The treatment was repeated 4 additional times on the same sample, with the intention of simulating 5 additional regenerations of the already commercially regenerated MCM-22. A 0.20 gram of this multiple regenerated catalyst, chopped to 1.6 mm length, was evaluated for 2-butene oligomerization using the same procedure described in Example 1. Representative data are shown in Tables 3-5.

Comparison of Catalyst Performance in a Fixed-Bed Reactor

Tables 1-3 compare catalyst performance at 50%, 70%, and 90% conversion of 2-butene. Table 4 further compares catalysts activity and deactivation rate. Relative activity of each catalyst is determined by measuring the first-order rate constant for 2-butene oligomerization at 170° C. relative to that of SPA catalyst. Deactivation rate is given as conversion drop observed per day per WHSV.

The data in Tables 3-5 show that, when compared to SPA at constant conversion level, MCM-22 provided comparable product selectivity and average branching. MCM-22 produced slightly more di-branched and less tri-branched octenes than those of SPA.

TABLE 3
Comparison of Catalyst Performance at Nominal 50% Conversion

| | Catalyst | | | | | |
|---|---|---|---|---|---|---|
| | | | Alumina- | | Regen. | Multiple |
| | | Binder-free | bound | Regen | Steamed | Regen |
| | SPA | MCM-22 | MCM-22 | MCM-22 | MCM-22 | MCM-22 |
| | Ex. Number | | | | | |
| | 1 | 2 | 3 | 4 | 5 | 6 |
| WHSV | 5.7 | 48.7 | 29.3 | 33.3 | 17.8 | 28.1 |
| TOS, day | 13.1 | 8.1 | 4.8 | 5.3 | 0.8 | 3.9 |
| Conversion % | 53.5 | 53.4 | 54.2 | 48.5 | 56.26 | 55.21 |

Product Selectivity, wt %

| | | | | | | |
|---|---|---|---|---|---|---|
| C₅₋₇ | 0.91 | 1.13 | 1.44 | 1.60 | 1.61 | 1.61 |
| C_{8═} | 80.71 | 82.94 | 79.58 | 78.16 | 75.27 | 77.57 |
| C₉₋₁₁ | 2.18 | 2.44 | 3.65 | 3.50 | 3.55 | 3.44 |
| C_{12═} | 14.65 | 11.45 | 12.94 | 13.01 | 14.81 | 13.11 |
| C_{16═} | 1.50 | 2.05 | 2.38 | 3.63 | 4.49 | 3.65 |
| C_{20═} | 0.05 | 0.00 | 0.02 | 0.12 | 0.22 | 0.62 |
| C₂₄₊ | 0.00 | 0.00 | 0.00 | 0.00 | 0.06 | 0.00 |
| Total | 100.00 | 100.00 | 100.00 | 100.00 | 100.00 | 100.00 |

Average Branching

| | | | | | | |
|---|---|---|---|---|---|---|
| Me/C8 | 1.98 | 1.97 | 1.98 | 1.99 | 1.98 | 1.99 |
| Me/C12 | 2.47 | 2.45 | 2.44 | 2.44 | 2.45 | 2.44 |

Octene Distribution, %

| | | | | | | |
|---|---|---|---|---|---|---|
| Linear | 0.08 | 0.04 | 0.06 | 0.03 | 0.02 | 0.02 |
| Mono- | 6.28 | 4.65 | 4.59 | 3.09 | 3.20 | 2.94 |
| branched | | | | | | |
| di-branched | 89.28 | 93.63 | 92.81 | 94.85 | 95.09 | 95.24 |
| tri-branched | 4.36 | 1.68 | 2.54 | 2.03 | 1.68 | 1.80 |
| Sum | 100.00 | 100.00 | 100.00 | 100.00 | 100.00 | 100.00 |

TABLE 4
Comparison of Catalyst Performance at Nominal 70% Conversion

| | Catalyst | | | | |
|---|---|---|---|---|---|
| | | Binder-free | Alumina-bound | Spent-Regen | Multiple Regen |
| | SPA | MCM-22 | MCM-22 | MCM-22 | MCM-22 |
| | Example Number | | | | |
| | 1 | 2 | 3 | 4 | 6 |
| WHSV | 4.0 | 38.1 | 16.3 | 6.7 | 19.0 |
| TOS, day | 11.5 | 5.9 | 6.3 | 8.8 | 0.8 |
| Conversion % | 70.7 | 70.4 | 68.7 | 73.3 | 66.3 |

Product Selectivity, wt %

| | | | | | |
|---|---|---|---|---|---|
| C₅₋₇ | 1.03 | 1.65 | 1.91 | 1.98 | 2.04 |
| C_{8═} | 77.98 | 77.41 | 71.52 | 71.14 | 70.55 |
| C₉₋₁₁ | 2.48 | 3.27 | 4.52 | 4.31 | 4.24 |
| C_{12═} | 16.85 | 14.25 | 17.21 | 16.38 | 16.34 |
| C_{16═} | 1.61 | 3.42 | 4.82 | 5.75 | 6.21 |
| C_{20═} | 0.02 | 0.00 | 0.02 | 0.40 | 0.56 |
| C₂₄₊ | 0.03 | 0.00 | 0.00 | 0.04 | 0.06 |
| Total | 100.00 | 100.00 | 100.00 | 100.00 | 100.00 |

Average Branching

| | | | | | |
|---|---|---|---|---|---|
| Me/C8 | 1.99 | 1.97 | 1.99 | 1.99 | 1.99 |
| Me/C12 | 2.47 | 2.44 | 2.43 | 2.43 | 2.44 |

Octene Distribution, %

| | | | | | |
|---|---|---|---|---|---|
| Linear | 0.08 | 0.06 | 0.06 | 0.03 | 0.03 |
| mono-branched | 6.19 | 5.38 | 4.68 | 3.72 | 3.50 |
| di-branched | 87.92 | 92.08 | 91.46 | 93.51 | 94.05 |
| tri-branched | 5.82 | 2.48 | 3.79 | 2.74 | 2.42 |
| Sum | 100.00 | 100.00 | 100.00 | 100.00 | 100.00 |

TABLE 5
Comparison of Catalyst Performance at Nominal 90% Conversion

| | Catalyst | | |
|---|---|---|---|
| | | Binder-free | Alumina-bound |
| | SPA | MCM-22 | MCM-22 |
| | Example Number | | |
| | 1 | 2 | 3 |
| WHSV | 2.2 | 19.2 | 13.1 |
| TOS, day | 20.8 | 3.9 | 0.1 |
| Conversion % | 92.48 | 88.1 | 80.4 |

Product Selectivity, wt %

| | | | |
|---|---|---|---|
| C₅₋₇ | 1.55 | 3.28 | 2.54 |
| C₈₌ | 67.62 | 64.94 | 61.52 |
| C₉₋₁₁ | 3.68 | 5.11 | 5.92 |
| C₁₂₌ | 23.44 | 18.67 | 21.22 |
| C₁₆₌ | 3.41 | 7.34 | 8.21 |
| C₂₀₌ | 0.30 | 0.66 | 0.59 |
| C₂₄₊ | 0.00 | 0.00 | 0.00 |
| Total | 100.00 | 100.00 | 100.00 |

Average Branching

| | | | |
|---|---|---|---|
| Me/C8 | 2.05 | 1.98 | 2.01 |
| Me/C12 | 2.47 | 2.41 | 2.43 |

Octene Distribution, %

| | | | |
|---|---|---|---|
| Linear | 0.12 | 0.12 | 0.09 |
| mono-branched | 6.63 | 6.85 | 5.08 |
| di-branched | 81.67 | 87.87 | 88.65 |
| tri-branched | 11.58 | 5.16 | 6.19 |
| Sum | 100.00 | 100.00 | 100.00 |

Data in Table 6 show that MCM-22 catalysts are more active than SPA: nine times more active with binderless MCM-22 and five times with alumina-bound MCM-22. A comparison of deactivation rate shows that MCM-22 catalysts are significantly more stable than SPA. The data also show that spent-regenerated and multiple-regenerated MCM-22 catalysts have similar performance as fresh MCM-22.

TABLE 6
Comparison of Catalyst Activity and Stability

| | Catalyst | | | | | |
|---|---|---|---|---|---|---|
| | | Binder- | Alumina- | | Regen | Multiple |
| | | free | bound | Regen | Steamed | regen. |
| | SPA | MCM-22 | MCM-22 | MCM-22 | MCM-22 | MCM-22 |
| | Example No. | | | | | |
| | 1 | 2 | 3 | 4 | 5 | 6 |
| 1st-Order Rate Constant | 4.3 | 40.3 | 20.9 | 20.7 | 14.8 | 20.6 |
| Relative Activity | 1.0 | 9.4 | 4.9 | 4.8 | 3.4 | 4.8 |
| Deact. Rate | 0.90 | 0.07 | 0.02 | 0.03 | 0.60 | 0.04 |
| (% Conv. Drop/day/WHSV) | | | | | | |

### EXAMPLE 8

### Propylene Oligomerization in with a Spent-Regenerated MCM-22

A Spent-Regenerated MCM-22 catalyst (SiO₂:Al₂O₃ 25:1) in the form of a chopped 1.5 mm extrudate 1.5 mm long, alpha value 330, was evaluated for propylene oligomerization in a 9 mm outside diameter downflow stainless steel three-zone reactor. Conditions used included a nominal temperature of 170 C., pressure from a nominal 5270-6550 kPaa (750-950 psig) and a WHSV of 8.0 hr⁻¹. The propylene was first flowed over an alumina guard bed to sorb impurities.

Representative data are shown in Table 7 below.

TABLE 7
Propylene Oligomerization

| | Sample No. | | | | | |
|---|---|---|---|---|---|---|
| | 1 | 2 | 3 | 4 | 5 | 6 |
| Time on | 31 | 55 | 79 | 103 | 151 | 175 |
| Stream, hrs. | | | | | | |
| Conditions | | | | | | |
| Temp, ° C. | 169 | 169 | 169 | 170 | 170 | 170 |
| Press, kPag | | | | | | |
| Flow Rate, | 5.43 | 5.43 | 5.43 | 5.43 | 5.43 | 5.43 |
| ml · hr^{.-1} | | | | | | |
| WHSV, hr^{.-1} | 8.0 | 8.0 | 8.0 | 8.0 | 8.0 | 8.0 |
| Conversion, % | 97.83 | 97.54 | 96.32 | 95.85 | 92.50 | 90.41 |
| Selectivity, % | | | | | | |
| C5-C12 | 79.27 | 85.48 | 88.63 | 89.94 | 93.01 | 94.83 |
| C3 | 0.29 | 0.48 | 0.35 | 0.30 | 0.27 | 0.22 |
| C4═, C4══ | 0.09 | 0.12 | 0.11 | 0.12 | 0.10 | 0.10 |
| C4 | 0.11 | 0.16 | 0.13 | 0.10 | 0.07 | 0.06 |
| C5 | 0.46 | 0.55 | 0.50 | 0.47 | 0.39 | 0.34 |
| C6 | 3.67 | 3.76 | 3.98 | 4.44 | 5.63 | 6.59 |
| C7-C8 | 5.45 | 6.23 | 6.06 | 6.05 | 5.95 | 5.80 |
| C9 | 29.14 | 34.66 | 37.80 | 41.71 | 45.55 | 47.77 |
| C10-C11 | 11.44 | 11.54 | 11.70 | 11.22 | 11.08 | 10.08 |
| C12 | 29.10 | 28.75 | 28.60 | 26.04 | 24.41 | 23.44 |
| C15 | 10.20 | 8.63 | 6.71 | 6.10 | 4.69 | 3.66 |
| C16+ | 10.04 | 5.13 | 4.08 | 3.45 | 1.86 | 1.13 |
| Total | 100.00 | 100.00 | 100.00 | 100.00 | 100.00 | 100.00 |
| Me/C6 | 1.01 | 1.02 | 1.01 | 1.00 | 1.00 | 0.99 |
| Me/C9 | 2.47 | 2.47 | 2.48 | 2.48 | 2.48 | 2.48 |
| Me/C12 | 2.55 | 2.54 | 2.54 | 2.61 | 2.62 | 2.65 |

The results reported in Table 7 show that the catalyst retains its activity and selectivity for propylene conversion over extended periods of time with good selectivity to products below C₁₂ and moderately good selectivity to gasoline boiling range products no higher than C₁₂.

### EXAMPLE 9

### Butene Oligomerization/Alkylation over MCM-22

A C4 feed comprising isomeric butenes and isobutane was reacted over a regenerated MCM-22 catalyst in a laboratory scale reactor unit at 3790 kPag (550 psig) at temperatures from 124° to 236° C. (256 to 456° F.), using one, two or three reactors in sequence under isothermal (I) or adiabatic (A) conditions. The feed composition was as shown in Table 8 below. The three reactors contained, respectively, 4.84 g, 17.74 g. and 27.42 g of the catalyst.

TABLE 8
C4 Feed Composition

| | |
|---|---|
| 1-butene | 10% |
| cis 2-butene | 9% |
| trans-2-butene | 9% |
| isobutene | 11% |
| isobutane | balance |
| butylmercaptan | 15 ppm |
| water | 250 ppm |

The results are shown in Table 9 below. Selectivities are reported consistently on a C8+basis.

TABLE 9
C4 Olefin/Paraffin Conversion

| | Days on Stream | | | | | | | |
|---|---|---|---|---|---|---|---|---|
| | 5.2 | 7.7 | 8.9 | 11.5 | 15.8 | 20.0 | 21.6 | 25.8 |
| Operation | I | A | A | A | I | A | A | A |
| LHSV | 15.6 | 15.6 | 15.6 | 15.6 | 3.74 | 1.65 | 1.65 | 1.65 |
| Reactor 1 | | | | | | | | |
| Inlet T (C.) | 124 | 128 | 131 | 139 | 140 | 147 | 160 | 186 |
| Outlet T (C.) | 129 | 136 | 138 | 144 | 146 | 161 | 178 | 209 |
| Reactor 2 | | | | | | | | |
| Inlet T (C.) | N/A | N/A | N/A | N/A | 144 | 156 | 177 | 207 |
| Outlet T (C.) | N/A | N/A | N/A | N/A | 156 | 171 | 189 | 217 |
| Reactor 3 | | | | | | | | |
| Inlet T (C.) | N/A | N/A | N/A | N/A | N/A | 173 | 186 | 213 |
| Outlet T (C.) | N/A | N/A | N/A | N/A | N/A | 191 | 205 | 236 |
| Conversions | | | | | | | | |
| Overall olefin | 17% | 23% | 25% | 27% | 56% | 64% | 80% | 90% |
| (C4) convsn | | | | | | | | |
| 1+iso conversion | 31% | 40% | 43% | 45% | 87% | 93% | 95% | 97% |
| cis 2 conversion | 4% | 7% | 8% | 11% | 40% | 56% | 73% | 87% |
| trans 2 | 0% | 1% | 1% | 1% | 2% | 8% | 50% | 78% |
| conversion | | | | | | | | |
| iso-butane | 0% | 0% | 0% | 0% | 0% | 0% | 13% | 40% |
| conversion | | | | | | | | |
| Product camp | | | | | | | | |
| (wt. %) | | | | | | | | |
| 1+ Iso | 14.1 | 12.6 | 12.0 | 11.4 | 2.7 | 1.5 | 1.2 | 0.7 |
| Cis 2 | 9.0 | 8.7 | 8.6 | 8.3 | 5.7 | 4.2 | 2.7 | 1.2 |
| Trans 2 | 9.5 | 9.3 | 9.4 | 9.2 | 9.3 | 8.8 | 5.1 | 2.1 |
| C8s | 7.1 | 8.4 | 9.0 | 9.2 | 12.6 | 11.4 | 12.7 | 23.5 |
| C12+ | 0.6 | 1.0 | 1.3 | 1.8 | 3.6 | 4.1 | 5.8 | 11.0 |
| C16+ | 0.03 | 0.1 | 0.1 | 0.1 | 1.6 | 2.2 | 5.3 | 4.3 |
| C8 Selectivity | 92% | 88% | 86% | 83% | 71% | 65% | 53% | 61% |
| C12 Selectivity | 8% | 11% | 12% | 16% | 20% | 23% | 24% | 28% |
| C16 Selectivity | 0% | 1% | 1% | 1% | 9% | 12% | 22% | 11% |
| C12-C13 (wt. %) | 0.60 | 1.01 | 1.26 | 1.76 | 3.65 | 4.09 | 4.67 | 11.17 |
| C14-C15 (wt. %) | — | — | 0.00 | 0.00 | 0.12 | 0.04 | 0.04 | 0.04 |
| C16+ (wt. %) | 0.04 | 0.1 | 0.1 | 0.1 | 1.7 | 2.1 | 2.3 | 4.1 |

The results reported in Table 9 show that C8 selectivity increases at the highest olefin conversion levels with isobutane conversion increasing at a somewhat lower level indicating that alkylation of butene with isobutane is occurring at such levels.

### EXAMPLE 10

### Comparison with SPA Polymerization Product

A refinery FCC gas stream containing olefins up to C₄ was polymerized over an SPA catalyst to produce a gasoline boiling range product which was then hydrotreated at 65° C. (150° F.) and 93° C. (200° F.) over a Pt/Pd hydrotreating catalyst at 2410 kPag (3560 psig), 2 hr⁻¹ WHSV, 5:1 hydrogen/liquid ratio, to saturate olefins. The hydrotreated product was then analyzed for composition and the octane ratings (RON, MON) were determined by engine test. The same olefinic stream was also polymerized over an MCM-22 catalyst in a unit of the same configuration and the same determination made. The simulated distillation data (ASTM D 2887) are given in Table 10 below for the polymerized products and the hydrotreated (93° C.) products. The results of the octane testing are shown in Table 11 below. The hydrotreating technique was used to demonstrate more clearly the extent of branching in the two polymerization products: hydrotreatment saturates the olefins so that their contribution to product octane is eliminated with the differences remaining then being attributable to the branching of the paraffin chains, the product being essentially free of aromatics.

TABLE 10
Simulated Distillation, ° C.

| | | | Saturation | Saturation |
|---|---|---|---|---|
| | | | Product of | Product of |
| SimDis, | | MCM-22 | sPA Feed | MCM-22 |
| Pct. Off | sPA Product | Product | (93° C.) | Feed (93° C.) |
| 0.5 | −0.1 | −2.4 | 16.4 | −4.2 |
| 10 | 55.7 | 70.8 | 79.0 | 85.2 |
| 20 | 88.3 | 95.8 | 92.9 | 99.2 |
| 30 | 101.2 | 111.7 | 104.8 | 109.7 |
| 40 | 113.9 | 122.7 | 112.6 | 114.9 |
| 50 | 125.4 | 125.4 | 130.6 | 124.4 |
| 60 | 143.9 | 139.8 | 148.0 | 139.1 |
| 70 | 161.8 | 158.6 | 159.4 | 159.2 |
| 80 | 176.4 | 182.4 | 174.0 | 184.0 |
| 90 | 199.6 | 225.7 | 200.1 | 226.8 |
| 99.5 | 274.8 | 348.6 | 329.7 | 343.0 |

TABLE 11
SPA Product Octane Comparison

| | Sp. | | | | | |
|---|---|---|---|---|---|---|
| | Grav. | API | Br₂No | RON | MON | (R + M)/2 |
| MCM-22 Product | 0.7373 | 60.4 | 106.3 | 96.8 | 82.7 | 89.75 |
| Hydrotreated 65° C. | 0.7446 | 58.5 | 63.5 | 92.9 | 83.2 | 88.05 |
| Hydrotreated 93° C. | 0.7426 | 59 | 49.3 | 90 | 82.8 | 86.4 |
| SPA Product | 0.7296 | 62.4 | 113.7 | 96.2 | 82.2 | 89.2 |
| Hydrotreated 65° C. | 0.7315 | 61.9 | 58.9 | 89.1 | 82.3 | 85.7 |
| Hydrotreated 93° C. | 0.7322 | 61.8 | 51.5 | 86.2 | 81.5 | 83.85 |

The results in Table 11 show that the product from the MCM-22 polymerization initially has octane numbers which are slightly higher than those of the SPA product but upon hydrogenation to remove olefins, a moderate octane credit relative to the SPA product is consistently maintained, indicating that the MCM-22 product is more highly branched than the product from the SPA polymerization. In addition, the bromine number is less sensitive.

### EXAMPLE 11

### Reaction Product

A feed containing iso-butane and mixed butene isomers was passed into a three-reactor unit which could be operated under isothermal or adiabatic conditions containing a supported MCM-22 zeolite catalyst. The feed composition is given in Table 12 below. In successive runs, one, two or three of the reactors were used to simulate the operation of a chamber type unit. Reaction temperatures (first reactor inlet) varied from 124° C. (256° F.) to 186° C. (367° F.). The product compositions are given in Table 13 below together with the conditions for present in the unit.

The results indicate that at more severe conditions not only is polymerization taking place but also a degree of isobutene alkylation, as evidenced by the conversion of the iso-butane towards the end of the run period with three series reactors.

TABLE 12
Mixed C4 Feed

| | |
|---|---|
| Isobutane, wt. pct. | 60.2 |
| 1-C4= plus iso C4=, wt. pct. | 21.0 |
| Cis 2-C4=, wt. pct. | 9.4 |
| Trans 2-C4=, wt. pct. | 9.4 |
| Water, ppmw | 250.0 |
| Butyl mercaptans, ppmw | 15.0 |

TABLE 13
Butane/Butene Conversion

| | Days on Stream | | | | | | |
|---|---|---|---|---|---|---|---|
| | 5.2 | 7.7 | 8.9 | 15.8 | 20.0 | 21.6 | 25.8 |
| LHSV (hr − 1) | 15.6 | 15.6 | 15.6 | 3.74 | 1.65 | 1.65 | 1.65 |
| Pressure (kPag) | 3792 | 3792 | 3792 | 3792 | 3792 | 3792 | 3792 |
| Product | | | | | | | |
| Compositions | | | | | | | |
| 1C4═ + isoC4═ | 14.1 | 12.6 | 12.0 | 2.7 | 1.5 | 1.2 | 0.7 |
| Cis 2-C4═ | 9.0 | 8.7 | 8.6 | 5.7 | 4.2 | 2.7 | 1.2 |
| Trans C4═ | 9.5 | 9.3 | 9.4 | 9.3 | 8.8 | 5.1 | 2.1 |
| C8 | 7.1 | 8.4 | 9.0 | 12.6 | 11.4 | 12.7 | 23.5 |
| C12+ | 0.6 | 1.0 | 1.3 | 3.6 | 4.1 | 5.8 | 11.0 |
| C16+ | 0.03 | 0.1 | 0.1 | 1.6 | 2.2 | 5.3 | 4.3 |
| Conditions | | | | | | | |
| Reactor 1 | | | | | | | |
| Inlet Temp, C. | 124 | 128 | 131 | 140 | 147 | 160 | 186 |
| Outlet Temp, C. | 129 | 136 | 138 | 146 | 161 | 178 | 209 |
| R1 ΔT, C. | 4 | 7 | 7 | 6 | 14 | 18 | 23 |
| Reactor 2 | | | | | | | |
| Inlet Temp, C. | | | | 144 | 156 | 177 | 207 |
| Outlet Temp, C. | | | | 156 | 171 | 189 | 217 |
| R2 ΔT, C. | | | | 12 | 15 | 12 | 10 |
| Reactor 3 | | | | | | | |
| Inlet Temp, C. | | | | | 173 | 186 | 213 |
| Outlet Temp, C. | | | | | 191 | 205 | 236 |
| R3 ΔT, C. | | | | | 18 | 19 | 23 |
| Av. Bed Temp, C. | 127 | 133 | 136 | 150 | 174 | 188 | 216 |
| Overall ΔT, C. | 4 | 7 | 7 | 18 | 47 | 51 | 56 |
| Isothermal/Adiabatic | I | A | A | I | A | A | A |
| Steady State | N | Y | Y | N | Y | Y | Y |
| Conversions | | | | | | | |
| Overall C4 olefin | 17 | 23 | 25 | 56 | 64 | 80 | 90 |
| 1C4═ + isoC4═ | 31 | 40 | 43 | 87 | 93 | 95 | 97 |
| Cis 2C4═ | 4 | 7 | 8 | 40 | 56 | 73 | 87 |
| Trans 2C4═ | 0 | 1 | 1 | 2 | 8 | 50 | 78 |
| Isobutane | 0 | 0 | 0 | 0 | 0 | 13 | 40 |
| C8 Selectivity (C8+ | 92 | 88 | 86 | 71 | 65 | 53 | 61 |
| basis) | | | | | | | |
| C12 Selectivity | 8 | 11 | 12 | 20 | 23 | 24 | 28 |
| (C8+ basis) | | | | | | | |
| C16 Selectivity | 0 | 1 | 1 | 9 | 12 | 22 | 11 |
| (C8+ basis) | | | | | | | |
| C12-C13, wt. pct. | 0.60 | 1.01 | 1.26 | 3.65 | 4.09 | 4.67 | 11.17 |
| C14-C15, wt. pct. | — | — | 0.00 | 0.12 | 0.04 | 0.04 | 0.04 |
| C16+, wt. pct. | 0.04 | 0.1 | 0.1 | 1.7 | 2.1 | 2.3 | 4.1 |

1. A method for the conversion of an SPA olefin oligomerization process unit which includes a reactor in which light olefin feed is oligomerized to form gasoline boiling range hydrocarbon product, which conversion method converts the SPA unit to operation with a molecular sieve based olefin oligomerization catalyst, comprising withdrawing solid phosphoric acid [SPA] catalyst from the unit and loading an olefin condensation catalyst comprising an MWW zeolite material into the reactor of the process unit.

2. A method according to claim 1 in which the MWW zeolite material comprises a zeolite of the MCM-22 family.

3. A method according to claim 2 in which the olefin condensation catalyst comprises a regenerated zeolite of the MCM-22 family.

4. A method according to claim 1 in which the olefin condensation catalyst comprises a self-bound zeolite of the MCM-22 family.

5. A method according to claim 1 in which a light olefinic feed selected from ethylene, propene, butene, and mixtures of these olefins is oligomerized over the zeolite catalyst at a temperature from 100 to 300° C. and a pressure of not greater than 7000 kPag.

6. A method according to claim 1 in which the olefin feed is processed over the zeolite catalyst for a cycle duration between successive regenerations of not less than six months.

7. A method according to claim 1 in which the reaction is carried out in a reactor comprising a plurality of fixed beds of the olefin condensation catalyst with diluent injected between the beds.

8. A process for the production of high octane, gasoline boiling range blend component by the condensation of light olefins in the C₂-C₄ range produced by the catalytic cracking of a petroleum feedstock in a fluid catalytic cracking unit, comprising passing the olefinic feed to a fixed bed of an olefin condensation catalyst comprising as the active catalytic component, an MWW zeolitic material at a temperature from 100 to 300° C., a pressure not greater than 7000 kpa, and a space velocity of not more than 50 WHSV [hour⁻¹].

9. A process according to claim 8 in which the average branching of the C₅-200° C. product is at least 1.8 [ME/C8].

10. A process according to claim 9 in which the average branching of the C₅-200° C. fraction is at least 2.25 [ME/C12].

11. A process according to claim 8 in which the feed comprises ethylene, propylene or butane or mixtures thereof.

12. A process according to claim 8 in which the feed has a water content of 300 to 800 ppmw.

13. A process according to claim 8 in which the feed has a water content less than 200 ppmw.

14. A process according to claim 8 in which the octene components of the C₅-200° C. product comprise at least 85 weight percent di-branched C₈ hydrocarbons.

15. A process according to claim 14 in which the octene components of the C₅-200° C. fraction comprise at least 88 to 96 weight percent di-branched C₈ hydrocarbons.

16. A process according to claim 8 in which the olefinic feed passed to the fixed bed of the condensation catalyst includes a recycled olefin oligomer product in the gasoline boiling range as diluent to remove heat of reaction.

17. A process according to claim 16 in which the olefin condensation reaction is carried out in a reactor comprising a plurality of fixed beds of the olefin condensation catalyst with a diluent comprising a recycled olefin oligomer product in the gasoline boiling range injected between the beds to remove heat of reaction.

18. A process according to claim 1 in which the feed includes a branched-chain paraffin which reacts with the olefin in the presence of the catalyst to form branched chain paraffins in the gasoline boiling range.

19. A process according to claim 18 in which the feed comprises butenes and isobutane.

20. A process according to claim 19 in which the butene reacts with the isobutane to form C₈ reaction products.

21. A method for the conversion of an SPA olefin oligomerization process unit which includes a reactor in which light olefin feed is oligomerized to form gasoline boiling range hydrocarbon product and producing gasoline boiling range product with extended catalyst cycle life, which conversion method converts the SPA unit to operation with a molecular sieve based olefin oligomerization catalyst, comprising withdrawing solid phosphoric acid [SPA] catalyst from the unit, loading an olefin condensation catalyst comprising an MWW zeolite material into the reactor of the process unit and producing a high octane rating, gasoline boiling range blend component by the catalytic oligomerization of light olefins in the C₂-C₄ range produced by the catalytic cracking of a petroleum feedstock in a fluid catalytic cracking unit, by passing the olefinic feed to a fixed bed of an olefin condensation catalyst comprising as the active catalytic component, an MWW zeolitic material at a temperature from 100 to 300° C., a pressure not greater than 7000 kPag, and a space velocity of not more than 30 WHSV [hour⁻¹].

22. A method according to claim 21 in which the fluid diluent comprises a paraffinic fluid including olefin oligomers.

23. A method according to claim 22 in which the fluid diluent comprises light C₃ and/or C₄ paraffins.

24. A method for the production of a gasoline boiling range product by the oligomerization of light C₂-C₄ FCC off-gas olefin feed which comprises oligomerizing the light olefin feed in the presence of an olefin condensation catalyst comprising an MWW zeolite to form gasoline boiling range hydrocarbon product and producing gasoline boiling material at a temperature from 100 to 300° C., a pressure not greater than 7000 kPag, and a space velocity of not more than 30 WHSV [hour⁻¹], the gasoline boiling range product including octene components in the C₅-200° C. fraction comprising at least 85 weight percent di-branched C₈ hydrocarbons.

25. A process according to claim 24 in which the octene components of the C₅-200° C. fraction comprise from 88 to 96 weight percent di-branched C₈ hydrocarbons.

26. A process according to claim 25 in which the di-branched C₈ hydrocarbons comprise at least 92 weight percent of all octene components of product.

27. A process according to claim 24 in which the tri-branched C₈ hydrocarbons comprise not more than 4 weight percent of all octene components of product.

28. A process according to claim 24 in which the feed has a water content of 300 to 800 ppmw.

29. A process according to claim 24 in which the feed has a water content below 200 ppmw.

30. A process according to claim 24 in which the average branching of the C₅-200° C. product is at least 1.8 [ME/C8].
